(19) United States
(12) Patent Application Publication
(10) Pub. No.: US 2024/0285334 A1
(43) Pub. Date: Aug. 29, 2024

(54) MEDICAL INTERVENTION DEVICE

(71) Applicant: GYRUS ACMI, INC. D/B/A OLYMPUS SURGICAL TECHNOLOGIES AMERICA, Westborough, MA (US)

(72) Inventor: William G. O'Neill, Maple Grove, MN (US)

(21) Appl. No.: 18/571,429

(22) PCT Filed: Jun. 29, 2022
(86) PCT No.: PCT/US2022/073266

§ 371 Date: Dec. 18, 2023

Related U.S. Application Data
(60) Provisional application No. 63/216,973, filed on Jun. 30, 2021.
(60) Provisional application No. 63/270,684, filed on Oct. 22, 2021.

(51) Int. Cl. A61B 18/14; A61B 17/29; A61B 18/00; A61B 90/00

(52) U.S. Cl. CPC A61B 18/1445; A61B 90/06; A61B 2017/2903; A61B 2017/292; A61B 2017/2947; A61B 2018/00172; A61B 2018/00184; A61B 2018/00595; A61B 2018/0091

(57) ABSTRACT

A medical intervention device may include a handheld device adapted for minimally invasive procedures. The device may include a handle that remains outside of the patient and an extension that extends from the handle into the patient and includes an end effector such as a forceps at a distal end. The mechanical operating relationship between the extension and the end effector may provide for motion about multiple intersecting axes thereby avoiding a need for compensating motion. In particular, yaw and pitch motion may be imparted independent of one another and avoiding a need for compensating motion of one axis for another. Moreover, the mechanical operating relationships of the device be configured for precisely mimicking the hand motion of the surgeon or other user and may include ratioed motion that provides for more precise motion at the end effector than at the handle and/or tremor control.

### CROSS-REFERENCE TO RELATED APPLICATIONS

The present application claims priority to U.S. Provisional Application No. 63/216,973 entitled Handheld Telemanipulator for Endoscopic or Laparoscopic Procedures and filed on Jun. 30, 2021 and U.S. Provisional Application No. 63/270,684 entitled Medical Intervention Device and filed on Oct. 22, 2021, the content of each of which is hereby incorporated by referenced in its entirety.

### TECHNICAL FIELD

The present application relates generally to a medical intervention device. More particularly, this document pertains generally to a handheld medical intervention device having a manipulable tool for endoscopic, laparoscopic, and/or electrosurgical procedures. Still more particularly, the present application relates generally to a handheld medical intervention device having a distal portion providing motion about multiple intersecting axes, independent and/or constant velocity motion about the axes, ratioed motion for precision, tremor reduction, battery power, and/or four degrees of freedom.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

Medical intervention devices may often include a pair of jaws that form a clamp or forceps or another manipulable tool may be arranged on the end of a relatively long slender extension with a handle. This type of medical intervention device may allow the manipulable tool to be placed through an access port in a patient and provide for operation of the tool in spaced apart relation to the handle. As such, a surgeon or other user may be able to perform a procedure on a patient via relatively small incisions, but with the tool capabilities of an open procedure with much larger incisions. As may be appreciated, separating the tool portion of the device from the handle portion of the device can result in less control, smaller ranges of motion, fewer degrees of freedom, and/or fewer tool capabilities. Moreover, the hand-eye coordination that a surgeon or other user is accustomed to for open procedures may be affected by separating the tool from the handle. This may occur due to the lack of an ability of the device to mimic the performance or motion of a conventional tool that is not separated from the handle.

While efforts have been made, particularly in the robotic space, to provide tools that suitably mimic conventional handheld tool motion, such tools rely on sophisticated computing devices and motors that provide for compensating motion amongst the several motors and features of the tool.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 is a partial cutaway isometric view of an example of portions of a handheld medical intervention device, according to one or more examples.

FIG. 2 shows a close-up view of distal portion of the handheld medical intervention device of FIG. 1.

FIG. 3 shows a side view of the portions of the device of FIG. 1.

FIG. 4 shows a view taken along the section C-C of FIG. 3.

FIG. 5 shows a view taken along the section E-E of FIG. 3.

FIG. 6 shows an exploded view of the distal portion of FIG. 2.

FIG. 7 shows an example of the handheld medical intervention device that includes or relies on one or more sensors or motors to move the end effector, according to one or more examples.

FIG. 8 is a perspective partial cutaway view of an example of portions of the handheld medical intervention device, according to one or more examples.

FIG. 9A is a perspective view of a working element in the form of a ball chain engaged with a notched gear, according to one or more examples.

FIG. 9B is a perspective view of an adjustment link on the ball chain of FIG. 9A, according to one or more examples.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

The present application, in one or more embodiments, relates to a medical intervention device. The device may be a handheld device adapted for endoscopic, laparoscopic, or other minimally invasive procedures. As such, the device may include a handle that remains outside of the patient and an extension that extends from the handle into the patient and includes an end effector such as a forceps at a distal end. The mechanical operating relationship between the extension and the end effector may provide for motion about multiple intersecting axes thereby avoiding a need for compensating motion and allowing for simpler operation than computer-controlled motion compensating systems. In particular, yaw and pitch motion may be imparted independent of one another and avoiding a need for compensating motion of one axis for another. Moreover, the mechanical operating relationship between the end effector and the extension and the mechanical operating relationship between the handle and the extension may be configured for precisely mimicking the hand motion of the surgeon or other user. Still further, the mimicking motion may be ratioed motion that provides for more precise motion at the end effector than at the handle and may also provide for tremor control. Still further advantages and benefits of the present medical intervention device may be apparent to those of skill in the art from a review of the detailed description below.

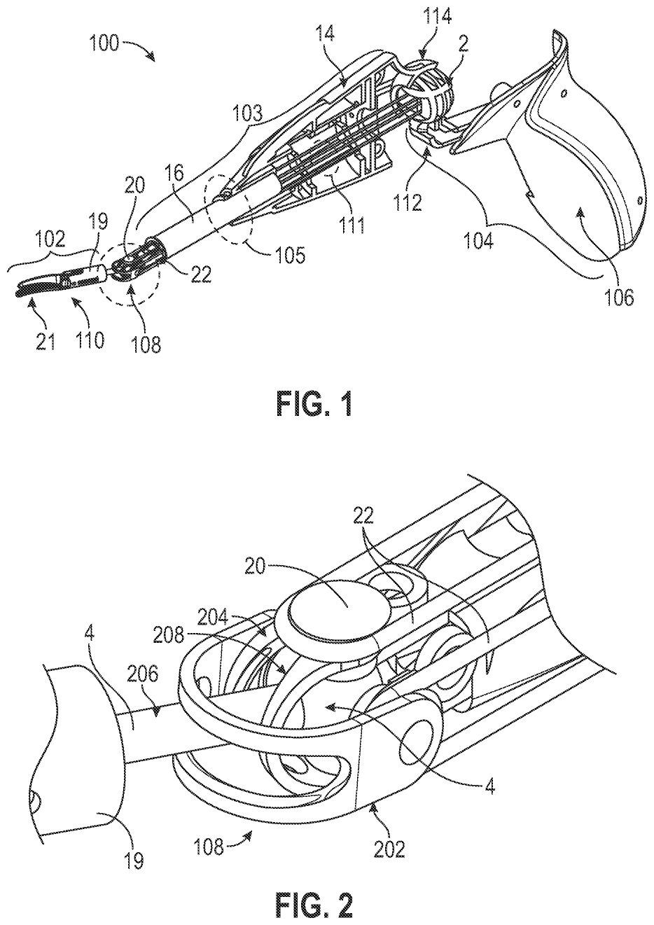

FIG. 1 shows a partial cutaway isometric view of an example of portions of a handheld medical intervention device 100, according to one or more examples. The device 100 may be adapted for performing a medical intervention by placing an end effector 110 through a trocar or other port on the surface of a patient and manipulating the end effector 110 to perform a procedure using a handle 106 positioned outside of the patient. As shown in FIG. 1, the handheld medical intervention device 100 may include a distal portion 102, a central portion 103, and a proximal portion 104. Each of these portions may be described in turn.

The proximal portion 104 may be configured to remain outside of a patient and to be manipulated by a surgeon or other user to control the distal portion 102 of the device 100. In one or more embodiments, the proximal portion 104 may include a handle 106. The handle 106 may be adapted for grasping and manipulation by a surgeon or other user and may include a pistol grip handle as shown. Alternatively, the handle 106 may include a rod-like handle aligned with the central portion or a u-shaped handle, a T-handle, or other types of handles may be provided. The handle 106 may include one or more actuators allowing the surgeon or other user to activate one or more features at the distal portion 102. For example, an actuator in the form of a plunger, lever, or other trigger may be provided for actuating jaws of a forceps or advancing and retracting a blade at the distal portion 102, for example. In addition, a button, switch, lever, or other trigger may be provided for actuating electrodes at the distal portion 102. Still other actuators may be provided depending on the nature of the end effector 110 provided at the distal portion 102.

The central portion 103 may extend from the proximal portion 104 to the distal portion 102. The central portion 103 may be configured to establish an offset distance between the proximal portion 104 and the distal portion 102 and, as such, provide for manipulation of one or more end effectors 110 at a distance spaced apart from the handle 106. The central portion 103 may also be configured to engage a port on the patient to establish a grounding point or interface relative to which the other parts and pieces of the device may move. As such, and as shown in FIG. 1, the central portion 103 may include a grounding element 14 and an extension element 16.

The grounding element 14 may include a relatively broad housing adapted to allow a user to handle and/or place the device 100 without imparting motion to the distal portion 102. The housing may surround working elements that extend between the proximal portion 104 and the distal portion 102 and, as such, may be substantially cylindrical, cone-shaped, or nose cone-shaped, for example. That is, in one or more embodiments, the relatively broad housing may taper to a narrower size as the housing extends distally and the taper may be a curved taper, for example. The housing may include a seating mechanism 105 at a distal end thereof that may be adapted to engage a trocar or other port on a patient. As such, the distal end of the housing may seat against or in the trocar or other port and establish a pivot point along the device 100 and about which the device 100 may be rotated for controlling the overall orientation of the device 100 relative to the patient. Moreover, the engagement of the distal end of the housing in the trocar or port may provide resistance to lateral swinging motion of the central portion 103 and resistance to twisting of the central portion 103 under motions of the handle 106 relative to the central portion and, as such, provide a relatively secure anchor point about which motion of the handle 106 may be performed to control features of the distal portion 102 and provide relative motion of the distal portion 102 to the central portion 103.

The extension element 16 of the central portion 103 may extend distally from the grounding element 14. In particular, the extension element 103 may overlap with the housing of the grounding element 14 to resist bending of the central portion 103 at the joint between the extension element 16 and the grounding element 14 and provide for a relatively stiff and continuous central portion 103. In one or more embodiments, for example, the housing of the grounding element 14 may include a cylindrical bore on a distal end with an annular seat at a proximal end of the bore. As shown, the extension element 16 may extend into the bore and be seated against the annular seat. The extension element 16 may engage the bore of the housing in an interference fit or adhesives, welding, or other securing systems may be provided. The extension portion 16 may extend distally from the grounding element 14 to the distal portion 102. The extension portion 16 may be sized and shaped for insertion through a trocar or other port on a patient and, as such, may have a relatively small diameter and may be substantially elongate. In one or more examples, the extension portion 16 may be a cylindrically shaped hollow shaft. While the extension portion 16 has been described as being relatively rigidly secured to the housing, in one or more examples, the extension portion 16 may more loosely engage the bore of the housing and, as such, be allowed or configured to rotate about a longitudinal axis of the device 100 relative to the housing of the grounding element 14.

The distal portion 102 may be arranged distal to the central portion 103 and may be adapted to move relative to the central portion 103 based on motion of the handle 106 relative to the central portion 103. Moreover, the distal portion 102 may be adapted to engage, grasp, cut, cauterize, or otherwise interface with patient tissue, vessels, organs, or other patient features. To this end, the distal portion 102 may include a base 19 and one or more end effectors 110 arranged on the base 19. As shown in FIG. 1, for example, the distal portion 102 may include a base portion 19 having a pair of forks for supporting a forceps. That is, for example, the base portion 19 may include a substantially cylindrical body having a notch cut out of a distal end thereof to form a pair of forks. An end effector 110 including a forceps may be mounted between the forks. The forceps may include a pair of jaws 21 where, for example, a bottom jaw is a stationary jaw and a top jaw is an articulating jaw. Alternatively, both jaws 21 may be articulating jaws. A pin or other laterally extending element may extend between the forks of the base 19 and establish a pivot axis for the top jaw or both jaws, as the case may be. In one or more examples, the jaw or jaws may include electrodes that may be energized to seal or cauterize tissues, vessels, or other patient features. While an end effector 110 in the form of a forceps has been described, a cutting blade may alternatively be provided or both may be provided. Still other types of end effectors 110 may be provided.

With the main structures of the device described, the relative motion of the several structures may now be discussed. That is, for example, the proximal portion 104 may be connected to the central portion 103 at an actuator interface 112 and the distal portion 102 may be connected to the central portion 103 at a manipulation interface 108. Each of these interfaces 112/108 may be described in turn.

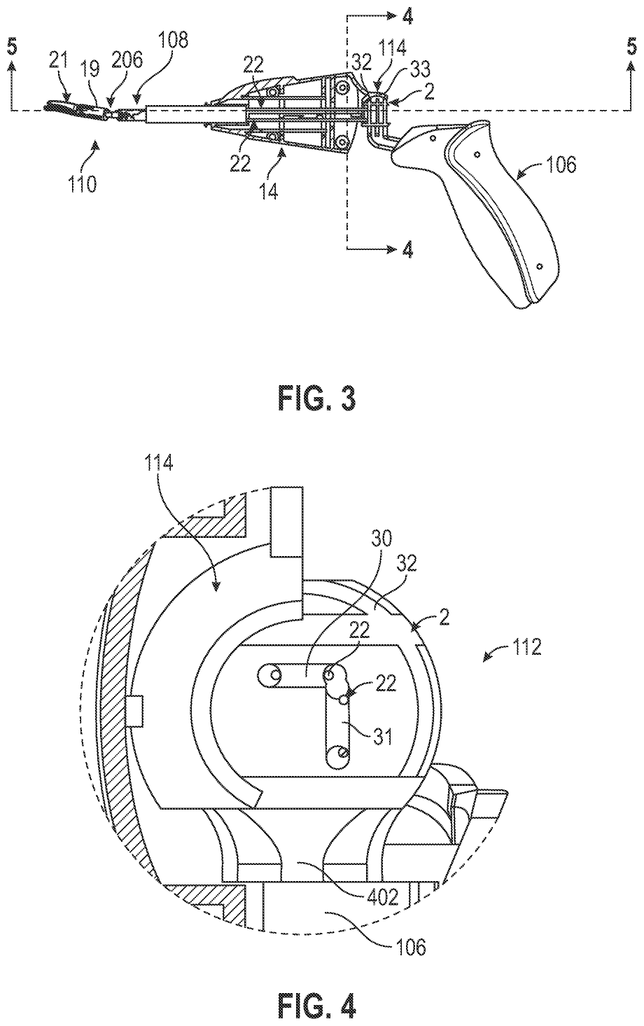

The actuator interface 112 may be configured to operably couple the proximal portion 104 to the central portion 103 and the distal portion 102. In particular, and with respect to the central portion 103, the actuator interface 112 may be configured to allow rotation of the handle 106 relative to the central portion 103 about each of three orthogonal axes (e.g., pitch, yaw, and roll) that intersect at a common point. In one or more examples, the actuator interface 112 may include a ball 2 and socket 114 where the handle 106 is rigidly secured to a substantially spherically shaped ball 2 and the central portion 103 is rigidly secured to a corresponding substantially spherically shaped socket 114. The ball 2 may be arranged in the socket 114 and be adapted for rotation in the socket 114. In one or more examples, the socket 114 may be arranged on and/or integrated into the proximal end of the housing of the grounding portion 114 as shown, for example, in FIG. 3. Moreover, the ball 2 may be arranged on and/or integrated into the distal portion of the handle 106 as shown, for example, in FIG. 3. In one or more examples, the handle 106 may be rigidly secured to the ball with one or more standoffs or struts. The standoffs or struts may be relatively narrow and/or small elements extending from the handle 106 and may be configured to secure the ball 2 to the handle 106 and reduce or minimize interference with the socket 114. That is, the range of motion of the handle 106 about any of the three orthogonal axes may be limited to the extent the standoffs or struts interfere with the socket wall 114.

As shown by comparing FIGS. 1, 3, and 4, the socket wall may be substantially incomplete to allow the standoffs or struts to rotate with the ball 2 without engaging the wall of the socket 114. In one or more examples, the socket 114 may include an open bottom, an open distal side, and an open proximal side. As shown in FIG. 1 and with respect to the open proximal side, the socket 114 may extend slightly proximally from a vertical centerline of the socket 114 to inhibit proximal longitudinal motion of the ball 2 and maintain the ball 2 in the socket 114. For example, the socket 114 may extend approximately 20 to 30 degrees proximally from a vertical centerline. With respect to the open distal side, the socket 114 may extend slightly further distally than it does proximally. That is, as shown, the socket 114 may extend approximately 30 to 45 degrees distally from a vertical centerline. With respect to the open bottom and with reference to FIG. 4, the socket 114 may extend approximately 30 to 45 degrees downward from a horizontal centerline of the socket 114. As such, though the socket 114 is open on the distal, proximal, and bottom sides, the socket may encompass the ball 2 sufficiently on each of these sides to prevent dislodgment in all longitudinal directions. However, the open bottom and proximal sides may provide pathways for the standoffs or struts of the handle 106 to move about the socket 114 through substantially large ranges of motion. For example, with respect to rotation about a longitudinal axis (e.g., twisting or roll rotation), the handle 106 may be free to travel through a range of motion of approximately 90 degrees (e.g., 45 degrees to either side of a vertical centerline). With respect to rotation about a lateral horizontal axis (e.g., pitch rotation), the handle 106 may be free to travel through a range of motion of approximately 120 degrees (e.g., from a position where the handle 106 is rotated further clockwise as compared to the position in FIG. 3 to a position counterclockwise of the position shown in FIG. 3 and where the standoffs or struts extend proximally out of the socket). With respect to rotation about a vertical axis (e.g., yaw rotation), the handle 106 may be free to travel through a range of motion of approximately 90 degrees (e.g., 45 degrees to either side of a longitudinal centerline).

As mentioned, the actuator interface 112 may be configured to operably couple the proximal portion 104 to the central portion 103 and the distal portion 102. With respect to operable coupling to the distal portion 102 one or more working elements 111 may be coupled to the actuator interface 112 such that motion of the handle 106 is transmitted through the central portion 103 to the distal portion 102 to provide corresponding motion at the distal portion 102. One or more working elements 111 may be provided for each respective type of motion. That is, one or more working elements 111 may be provided for each of pitch, yaw, and roll such that pitch motion, yaw motion, or roll motion of the handle 106 causes corresponding pitch, yaw, and roll motion at the distal portion 102.

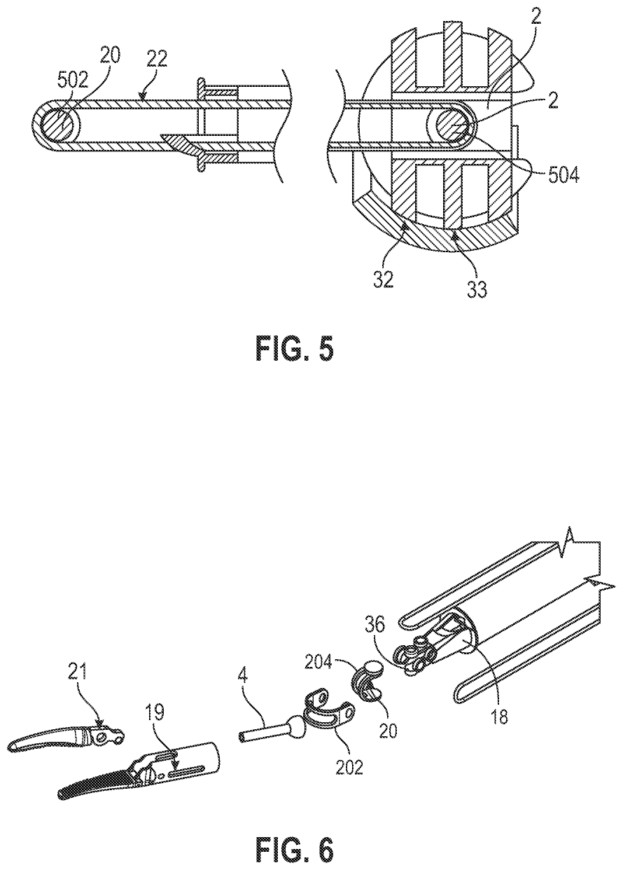

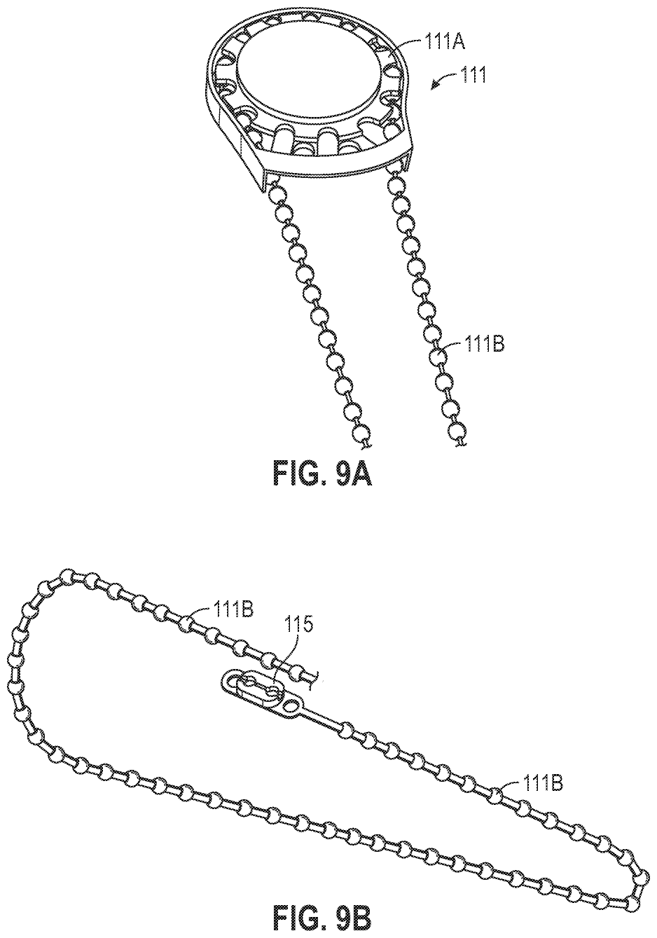

With respect to pitch and yaw motion of the handle 106 and the distal portion 102, a plurality working elements 111 in the form of tensile ties or loops may be provided to generate rotation at the distal portion 102 that reflects the rotation at the proximal portion 104. In one or more embodiments, and as shown in FIGS. 4 and 5, the ties may include flexible and/or elastic loops such as belts or chains, for example, and the loops may engage a circular surface 30/31 on the actuator interface 112 that may function like a fixed pulley or sprocket, respectively. As shown in FIG. 4, for example, the actuator interface 112 may include slots leading to generally circular or semicircular surfaces 30/31. The slots may be arranged in plane with the circular shapes defined by the circular or semicircular surfaces 30/31 and may be sized such that each end of the slot is generally tangential to the circular or semicircular surfaces 30/31 allowing the loops to pass into the actuator interface 112 and bend across the circular or semicircular surfaces 30/31 and return out of the actuator interface 112. In one or more examples, the circular or semicircular shapes defined by the surfaces 30/31 may be centered on respective axes of motion of the ball 2 of the actuation interface 112. That is, for example, the circular or semicircular surface 30/31 may define a circular shape having a center point and the center point may be arranged on an axis orthogonal to the circular shape and passing through the center point of the ball 2 of the actuation interface 112. The loops may frictionally engage the circular or semicircular surfaces 30/31 or, in the case of a chain, may engage teeth on the surface such that rotation of the ball 2 within the actuator interface 112 rotates the circular or semicircular surface 30/31 thereby rotating the loop and transmitting that rotation to the distal portion 102 via relative translation of each leg of the loop. One example of a chain system includes a ball chain 111B with a notched gear 111A as shown in FIG. 9A. As shown, the working element 111 may include a ball chain 111B that extends around a notched gear 111A arranged on or incorporated into the circular surface 30/31 and providing an anti-slip engagement between the working element 111 and the actuator interface 112 and/or the manipulation interface 108 (e.g., where a notched gear is provided on the manipulation interface 108 as well). In one or more examples as shown in FIG. 9B, an adjustment link 115 may be provided within the length of the ball chain 111B allowing the overall length and, thus, the tension or tightness of the chain 111B to be adjusted. As shown, the adjustment link 115 may include ball seats allowing the chain 111B to be lifted from the adjustment link 115, adjusted (e.g., tightened or loosened), and reseated in the ball seats of the adjustment link 115. In the case of elastic ties or loops, the ties or loops may absorb vibrations to avoid transmitting such vibrations to the distal portion 102. For example, the ties or loops may be an elastomeric or other elastic material that may assist with absorbing vibrations such as from tremors induced at the handle 106, for example. Still further, the elastic ties or loops may allow for maintaining tension in the ties or loops when rotation of the ball 2 causes the loops or ties to slacken, for example.

In one or more examples, the radius of curvature and, thus, the size or width of the circular or semicircular surfaces 30/31 may be selected relative to a corresponding circular or semicircular surface at the distal portion 102 to magnify or reduce the motion at the distal portion 102 relative to the motion of the handle 106. That is, for example, where a higher precision of motion at the distal portion 102 is desired, the size or width of the circular or semicircular surface 30/31 at the actuator interface 112 may be selected to be smaller than the size or width of the circular or semicircular surface at the distal portion 102. This is because angular motion at the actuator interface 112 will cause a particular amount of longitudinal motion of the respective legs of the loop which gets translated to the distal portion 102 where the longitudinal motion will cause a smaller amount of angular motion at the distal portion 102 (same amount of longitudinal motion along a larger circle results in smaller angular motion of the distal portion).

It is to be appreciated that while the working elements 111 for pitch and yaw have been said to include loops, the ties may be dead ended into the actuator interface 112 rather than looping through the actuator interface 112 and the same may be true at the distal portion 102. Moreover, cam surfaces may be provided along the actuator interface 112 on a proximal side of the ball 2, for example, that extend from one dead end to the other dead end. Similar cam surfaces may be provided at the manipulation interface 108 near the distal portion 102. The spacing between the dead ended ties may be adjusted and selected to provide the desired amount of precision or magnification of motion of the handle 106. Still further, while tensile elements such as flexible loops have been described, a more rigid element such as a wire or strut may extend from the actuator interface 112 to the distal portion 102 and may transfer tension and compression depending on the position of the wire or strut and the direction of rotation of the actuator interface 112. That is, rather than two tension ties, a single or multiple tension/compression members may be provided.

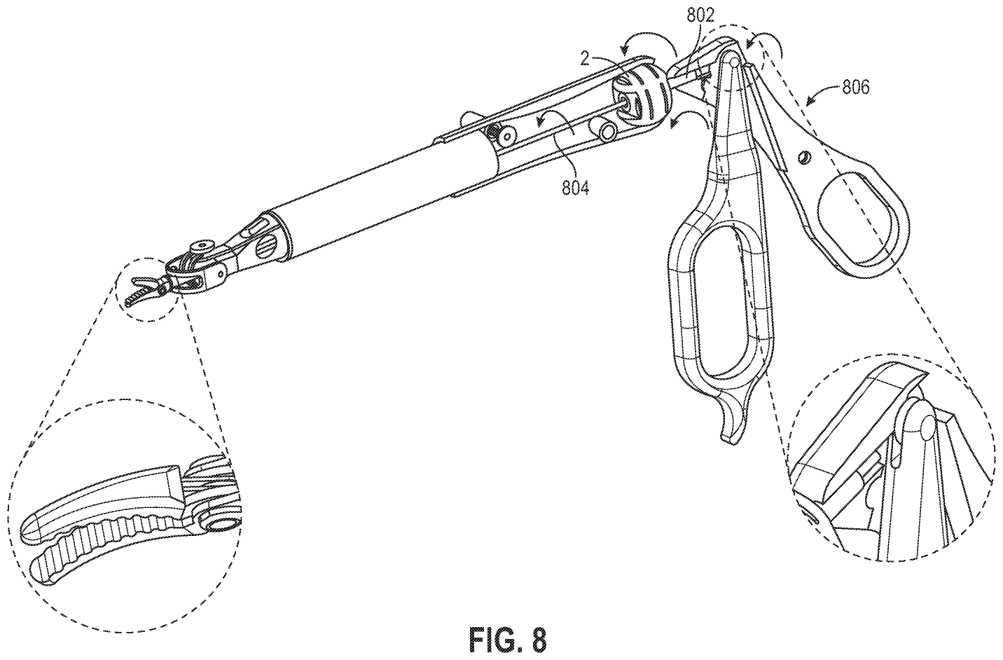

As mentioned, one or more working elements 111 may be provided for a respective type of motion including, each of pitch, yaw, and roll. With respect to roll, and with reference to FIG. 8, working element 111 in the form of a torque element 804 may be provided for transmitting roll motion of the handle 106 to the distal portion 102. As shown, a flexible torque tube, shaft, spring, coil, braided mesh, or other torque transmitting element 804 may extend distally from the actuator interface. In particular, as shown, the torque element 804 may be substantially rigidly secured to the handle 106 and may extend longitudinally and sleevably through the center of the actuator interface 112. Alternatively, the torque element 804 may be rigidly secured to the ball 2 of the actuator interface 112. In either case, twisting or roll motion of the handle 106 may induce twisting or roll motion of the torque element 804, which may transmit that roll motion to the distal portion 102. The torque element 804 may be flexible about axes orthogonal to the longitudinal axis of the torque element 804 to allow the torque element 804 to accommodate yaw or pitch motion of the handle 106 without kinking or breaking and while maintaining its ability to transmit roll motion along its length.

As mentioned, the distal portion 102 may be operably coupled to the central portion 103 with a manipulation interface 108. The manipulation interface may be configured to receive motion from the one or more working elements 111 extending through the central portion 103 and induce motion in the distal portion 102 that corresponds to motion at the proximal portion 104. That is, the handle 106 may be operable to rotate with 1, 2, or 3 degrees of rotational freedom about orthogonal axes that intersect at a point such as at the center of the actuation interface 112. Likewise, the manipulation interface 108 may provide for 1, 2, or 3 degrees of rotational freedom of the distal portion 102 about orthogonal axes that correspond with the orthogonal axes of the handle 106, but intersect at a different point such as the center of the manipulation interface 108. As such, control of the rotation of the distal portion 102 about 1, 2, or 3 orthogonal axes that intersect at a point may be provided by the system and such rotation may correspond with rotation of the handle 106. That is, for example, pitch motion of the handle 106 may cause corresponding pitch motion of the distal portion 102; yaw motion of the handle 106 may cause corresponding yaw motion of the distal portion 102, and roll motion of the handle 106 may cause corresponding roll motion of the distal portion 102. Moreover, and while the motion may correspond by being about a corresponding axis, the ratio of motion may be different to provide for higher levels of precision, for example. This system is advantageous by providing such corresponding motion without the need for computer compensation of motion. That is, since the motion is about axes that intersect at a common point, compensating motion due to motion about another axis may be avoided and sophisticated robotic compensating motion programming may likewise be avoided. Still further at least two of the rotational degrees of freedom about the common point at the manipulation interface may be independent of one another. That is, as discussed in more detail below, pitch and yaw motion may be independent of one another, which is to say that pitch motion does not adjust the yaw axis and yaw motion does not adjust the pitch axis. Pitch and yaw motion also do not adjust the roll axis. Roll motion, on the other hand, may have an effect on other axes by rotating the pitch and yaw axes with the roll axis and maintaining the orthogonal relationship between the pitch and yaw axes. This is in contrast to a universal joint, which may have only 1 degree of freedom that is independent of the other axes, for example.

With respect to pitch and yaw motion of the distal portion 102, reference is made to FIGS. 6 and 8. As shown, the manipulation interface 108 may include a turret, stand, or other support 18 arranged on the distal end of the extension element 16 of the central portion 103. The turret 18 may be configured to support pulleys or cam surfaces for interaction with the tension ties or loops to control pivoting motion of the distal portion 102 about a vertical axis (e.g., yaw) and pivoting motion about a laterally extending horizontal axis (e.g., pitch). In one or more embodiments, the turret 18 may be pyramid shaped such that it tapers slightly as it extends distally. The turret 18 may include sidewalls on each of 4 sides that extend distally to respective pivot points. As shown, a gimbal may be provided for each of the yaw and pitch motions of the distal portion and the gimbals may be supported at the pivot points of the walls of the turret 18. For example, the pitch gimbal 202 may be secured to pivot points of the turret 18 that are at the distal end of walls extending distally along the sides of turret 18. A yaw gimbal 204 may be secured to pivot points of the turret 18 that are at the distal end of walls extending distally along the top and bottom of the turret 18. The gimbals 202/204 may be substantially u-shaped elements having, for example a u-shaped bail with a slot passing therethrough and extending along the bail. Beyond the ends of the slots, the gimbals 202/204 may include pulleys, sprockets, or other features that are configured for engagement by the ties or loops allowing the relative translation of the legs of the ties or loops to rotate the pulley or sprocket and, as such, rotate the gimbal 202/204 about its respective yaw or pitch axis. That is, the pulley or sprocket may be fixed relative to the gimbal 202/204, but the gimbal 202/204 and corresponding pulley or sprocket may be pivotable relative to the turret 18. The pulleys/sprockets may be arranged on an outboard surface of the gimbal 202/204 or on an inboard surface. For example, as shown, the pulley/sprocket is arranged on an outboard surface of the yaw gimbal 204 and on an inboard surface of the pitch gimbal 202. As shown, the walls of the turret 18 may include windows allowing passage of the ties or loops through the walls of the turret 18 to reach the pulleys or sprockets arranged on the outside of the turret 18 at the pivot points. Alternatively, the turret 18 may be substantially rectangular or cylindrical and the pulleys or sprockets may be arranged on an inside thereof such that the loops or ties do not pass through the walls of the turret 18. In either case, motion of the handle 106 about its yaw or pitch axis may cause corresponding rotation of the actuation interface 112, which may rotate the respective tie or loop, which may rotate the respective gimbal 202/204 at the manipulation interface 108.

With respect to roll motion of the distal portion 102, the turret 118 may be pivotally coupled to the extension element 16 of the central portion 103 or the extension element 16 of the central portion 103 may be pivotally coupled to the grounding element 14. In either case, the turret 18 may be free to rotate about the longitudinal axis of the device 100 relative to the grounding element 14 of the device 100. As mentioned above, a working element 111 in the form of a torque element 804 may extend longitudinally along the device 100 such that rotation of the handle 106 induces rotation in the torque element 804. The torque element 804 may extend longitudinally through the grounding element 14 and the extension element 16 of the central portion 103 and may rotationally engage the turret 18. For example, radially extending spokes or struts may extend radially away from the torque element 804 to engage the turret 18. Accordingly, rotational motion of the handle 106 about a roll axis may induce rotational motion of the turret 18 about the roll axis. Moreover, the rotation of the handle 106 may rotate the actuation interface 112 a same or similar amount such that the loops or ties remain aligned with their respective anchor points at the actuation interface 112 and at the manipulation interface 108.

With reference to FIGS. 2 and 6, the manipulation interface 108 may also include a distal stem 4. The distal stem 4 may be configured to transmit yaw and pitch motion of the gimbals 202/204 to the distal portion 102 of the device 100. That is, the distal stem 4 may be pivotally supported in a distal end of the turret 18 by, for example, a core element 208 such as a stem ball, a spherical bushing, or a hoop. In one or more examples, the inner surfaces of the distal ends of the walls of the turret 18 may include cup-shaped seats 36 adapted to engage respective sides of the stem ball. The stem ball may be arranged in the cup-shaped seats 36. The distal stem 4 may be rigidly secured to the ball and extend generally distally from the ball and through each slot of the yaw gimbal 204 and the pitch gimbal 202. As such, when either or both of the yaw or pitch gimbal 202/204 rotate, the distal stem 4 may be caused to rotate a same amount while the stem ball remains seated in the cup-shaped seats 36. Moreover, the stem 4 may be a hollow element and the stem ball may include a bore allowing one or more jaw actuation wires or other working elements 111 to pass through. The torque element 804, however, may be secured to the stem ball such that rotational motion of the handle 106 about a roll axis is transmitted to the distal stem 4 and stem ball The distal portion 102 described above may be secured to the distal stem 4 so as to track with the orientation of the distal stem 4. For example, a base 19 of the distal portion 102 may be rigidly coupled to the stem 4 such that motion of the stem 4 is reflected by the same motion of the distal portion 102.

Still further degrees of freedom may be provided by the present device 100. That is, where, for example a forceps is arranged on the distal portion 102 and/or a cold cut blade is arranged thereon, one or more longitudinal actuators may extend from the handle 106 to the distal portion 102 to control the respective operation. For example, as mentioned above, the handle 106 may include an actuator in the form of a trigger. The trigger may function to pull on a longitudinal actuator or flexible wire that passes through the center of the actuation interface 112 through the central portion 103 and through the center of the manipulation interface 108, through the stem 4 and into the distal portion 102. The flexible wire may be pivotably coupled to an articulating jaw 21 of a forceps, for example, such that actuation of the trigger, opens and/or closes the articulating jaw 21. As similar system may be used to actuate a cold cut blade, for example. In one or more examples, the longitudinal actuator may be arranged within the torque element 804 and extend through the stem ball and the stem 4 to the distal portion 102.

It is to be appreciated that the substantially continuous nature of the roll system by way of securing the torque element to the stem ball may provide for a constant velocity joint. That is, unlike a universal joint, for example, the present design may establish an equal amount of rolling rotational displacement on the distal side of the manipulation interface as is induced on the proximal side of the manipulation interface.

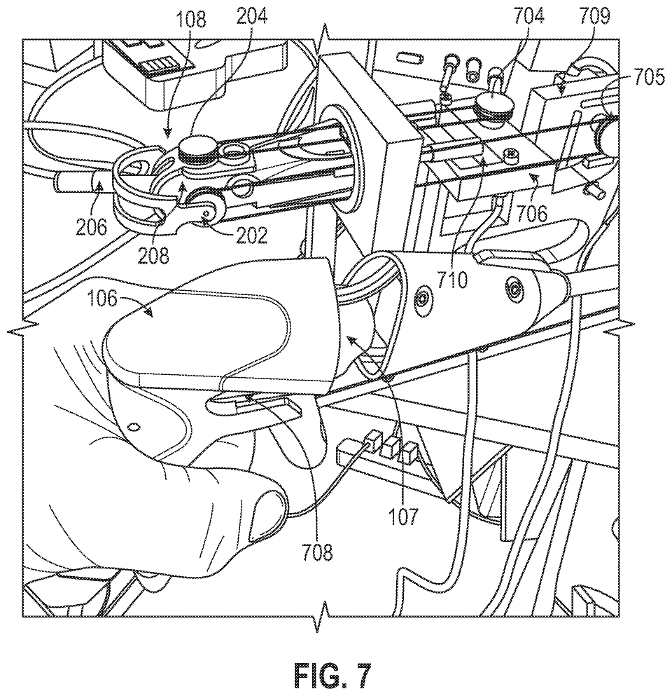

It is also to be appreciated that, while mechanical movement mechanisms have been described that do not include powered motion devices, powered motion devices such as servos or other mechanism may be provided with the system. For example, as shown in FIG. 7, the handle and actuation interface may be operably coupled to the manipulation interface via electrically powered servos, for example. That is, a handle that is the same or similar to the handle described above may be provided and an actuator interface that is the same or similar to the handle described above may also be provided. However, rather than mechanically coupling the actuator interface to the manipulation interface directly with working elements, sensors for sensing the motion of the handle may be provided. Moreover, servos may be provided for inducing motion in the distal portion via a manipulation interface that is the same or similar to the manipulation interface described above. That is, as shown, the servos may be configured to actuate pulleys or sprockets similar to the pulleys or sprockets shown and described with respect to the actuator interface above. Moreover, the pulleys or sprockets may be mechanically coupled to corresponding pulleys or sprockets on the manipulation interface. As such, motion of the handle may be sensed by a computing system and corresponding operation of a servo may cause corresponding motion in a respective pulley or sprocket, which motion may be transmitted to a corresponding pulley/sprocket at the manipulation interface to cause motion of the distal portion. This may, thus, function similar to the yaw and pitch motion of the above device, but be electromechanically controlled rather than mechanically controlled. With respect to roll motion, a similar set up may be provided where a roll servo is connected to a torque element extending to the manipulation interface to provide roll motion to the stem ball and stem and, thus, to the distal portion. It is noted that while the servos are shows as being coupled to the manipulation interface with tension ties or other working elements, the servos may also be directly connected to the manipulation interface by, for example, directly controlling the position of yaw and pitch gimbals, for example.

Where servos are provided for generating motion in the distal portion, one or more features may be provided. For example, while the mechanical system above was described as including vibration control via elastic elements in the system, the servo system may be configured to control vibration by filtering. That is, for example, the sensor data from the handle may be filtered by the computing system to remove tremor-like motion by filtering out high-frequency motion that is consistent with hand tremors, for example. Moreover, precision control may be provided by applying a reduction factor to the sensed motion of the handle when operating the servos. In one or more examples, such precision control may be provided for yaw and pitch, but not for roll. In other examples, the precision may be provided for yaw, pitch, and roll. Still other approaches and advantages may be provided. For example, while the device 100 has been described as a handheld device, a robotic approach may be used where a handle 106 is provided at a controller station of a user and the distal portion together with the manipulation interface, servos, and working elements may be provided as part of an operable robot. As such, one example of the present system may be as part of a robotic operation system where the servos, manipulation interface, distal portion, and potentially portions of the central portion are mounted on robotic arm, for example. The robot may be operated to insert the distal portion through a port on a patient and the handle may then be used to manipulate the distal portion via the sensors on the handle and computing system interpreting and translating those sensor signals to control the respective servos.

It is further to be appreciated that whether servos or electromechanical controls are provided or not, a power source may be provided for powering electrodes at the distal end. In one or more examples, the power source may be a corded power source or a battery power source may be provided. In one or more examples, the battery may be located in the handle and may be placed in electrical communication with the distal portion by way of electrical leads that pass through the center of the actuator interface, along the central portion and through the center of the manipulation interface to the distal portion. Electrodes may be present on the distal portion, for example on the jaws of a forceps, and the electrical leads may provide power to the electrodes. In one or more examples, a monopolar electrode may be provided or bipolar electrodes may be provided.

### EXAMPLES

As discussed in detail above, this document pertains generally, but not by way of limitation, to a handheld medical intervention device such as a telemanipulator for endoscopic procedures (which can refer generally to include endoscopic, arthroscopic, laparoscopic or similar minimally-invasive procedures), such as can include coincident (e.g., intersecting) pitch, yaw, and roll axes such as can provide three degrees of freedom articulation.

For comparison, in an approach in which the pitch, yaw, and roll axes are not coincident at a common intersection point, planes corresponding to individual axes are separated by a distance, which makes the smooth translation of the surgeon's wrist motion much more complicated than the present approach using a wrist mechanism such as can include coincident (e.g., intersecting) pitch, yaw, and roll axes such as can provide three degrees of freedom articulation. For example, in a non-coincident axes approach, it is complicated to rotate about a pitched axis, the offset distance between axes instead results in rotation around a cone defined by an angle resulting from the offset axes.

As may be apparent from the above detailed description, the present document describes, among other things, a handheld endoscopic or laparoscopic device such as for partial insertion into an orifice of or incision in a patient such as for treating a target within the patient. In an example, the device can include a distal portion, such as can be sized and shaped for insertion into the patient. The device can also include a proximal portion, such as can be connected to the distal portion. In an example, the proximal portion can include an external handle for a physician or other user to hold and support the proximal portion during insertion of the distal portion into the patient. A mechanical or electromechanical wrist such as the manipulation interface described can be located at the distal portion. The wrist can be configured to provide yaw, roll, and pitch motions about corresponding yaw, roll, and pitch axes, such as wherein each of the yaw, roll, and pitch axes are arranged to intersect one another at a common point. An actuator such as the actuation interface described above can be located at or near the handle. The actuator can be coupled to the wrist mechanism, such as for manipulating the wrist mechanism, such as for providing the yaw, roll, and pitch motions. An end effector can be located at the distal portion of the device and distal to the wrist. The end effector can provide at least yaw, roll, and pitch motions via the wrist in response to manipulation of the actuator by the user.

The present approach can position the pitch, roll and yaw axes to all coincide at a single point, such that the extra complication of non-coincident axes can be avoided and compensating motion issues may likewise be avoided. For example, a yaw element can pivotably rotate about the yaw axis without affecting the pitch or roll axes. This approach can help allow for a significant reduction in complication in that no sophisticated motors are required to control the motion of one axis to account for rotation of a different axis. The mechanism can be self-contained in a small, 8 mm package such as with small pulleys controlling the pitch and yaw axes. The third degree of freedom, roll, can use a flexible torque tube. Such a torque tube can manage torque using a spring which is covered in a flexible elastomer. The flexible elastomer can bend easily because the spring allows this flexion. However torque is resisted by the spring which is encased in the polymer. The roll axes then can either be driven by a torque catheter tube or a thin stainless steel tube in which cuts are made in the tubing to allow for bending but will transmit torque without deforming.

Thus, a sophisticated bipolar cut and seal device with a three degree of freedom wrist can be made with relatively simple and inexpensive components. This allows the device (or a portion thereof) to be disposed of after a single use. This can help avoid an approach in which wrist and end effectors are multiple use devices requiring cleaning and resterilization. The present approach can be advantageous for a device used to coagulate or cut tissue because these electrosurgical devices become encrusted with the patient's blood. The blood can be “baked” onto the cutting surfaces and is difficult to clean. The electrocautery can include small precise jaws with teeth, which may become covered in char and blood which will be difficult to clean. But the present approach can help provide an electrocautery device in which at least a portion of the device which contacts blood can be disposed of after use on a single patient.

The advantages of such a device for the surgeon are considerable, either as a monopolar or bipolar vessel sealing or other electrosurgical device, or as a device used by the surgeon in the non-dominant hand, such as can incorporate a non-dominant hand instrument such as one or more of a J-hook, spatula, forceps, and Lyons dissector. All of these instruments can be made more useful with a wrist which allows for three degrees of freedom. For example, for a colpotomy, a three dimensional wrist mechanism can be used to manipulate a J-hook to make a circular cut which would be difficult to do with a J-hook fixed to a rigid shaft. A Lyons dissector can be used as a needle driver to sew up the colpotomy edges. The device can be used to mimic the motion of a surgeon's hand suturing a wound, which would otherwise be difficult or impossible with a laparoscopic device limited by a pivot point at the trocar entry to the body cavity.

This present approach can solve many technical problems by providing a handheld instrument with electrosurgical cauterization capability but incorporating a wrist within the body cavity. The present handheld endoscopic or laparoscopic telemanipulator device may be ideal as a non-dominant hand device which allows surgeons the ability to access hard to reach surgical tissue. The wrist may be used to drive a needle and sew up a large cut. The two instruments can permit the surgeon to tie off a knot; a movement very difficult to do with two straight graspers.

A possible approach would have the surgeon use a bipolar vessel sealing and cutting instrument in their dominant hand but would use the present handheld medical intervention device to provide the non-dominant hand unparalleled flexibility. The device can be partially or completely disposed of after the surgery. The instrument does not require expensive capital equipment, extensive instrument cleaning and sterilization post-surgery and the learning curve should be fairly rapid since the wrist mimics the physician's wrist movement. This could open the door for advance surgical manipulation in all surgical procedures, such as can help avoid reserving time on an expensive surgical robot

While discussed in detail above, another way of describing the same or similar examples shown in the figures is as follows. FIG. 1 is a partial cutaway isometric schematic view of an example of portions of a handheld medical intervention device in the form of a telemanipulator device 100, such as for endoscopic or laparoscopic procedures. In the example of FIG. 1, the device 100 can include a handheld endoscopic or laparoscopic device 100, such as can include a portion that can be configured for partial insertion into an orifice of or incision in a patient, such as for treating a target within the patient. The device 100 can include a distal portion 102, such as can be sized and shaped for insertion into the patient. A proximal portion 104 can be connected to the distal portion 102. The proximal portion 104 can include an external handle 106. The handle 106 can be sized and shaped for a physician or other user to grip or hold, such as to at least one of support, orient, or direct the proximal portion 104 during insertion of the distal portion 102 into the patient. The distal portion 102 of the device 100 can include a mechanical or electromechanical manipulation interface or wrist mechanism 108. The wrist 108 can be configured to provide yaw, roll, and pitch motions, such as of an end effector 110, such as about corresponding yaw, roll, and pitch axes, wherein each of the yaw, roll, and pitch axes are arranged to intersect one another, such as explained herein. An actuator or actuation interface 112 can be located at or within the handle 106, or can be coupled thereto. The actuator 112 can be coupled to the wrist 108 (e.g., via one or more cables or pulleys) such as for manipulating the wrist 108 such as for providing the yaw, roll, and pitch motions of the wrist 108 and, therefore, of the end effector 110. The end effector 110 can be located at the distal portion 102 of the device 100, such as distal to the wrist 108. The end effector 110 can be configured to be capable of providing at least yaw, roll, and pitch motions via the wrist 108, such as in response to manipulation of the actuator 112 by the user, such as via grasping and moving or orienting the handle 106.

FIG. 1 shows an example of a mechanical version of a device 100, such as can be configured as a three dimensional (3D) Lyons dissector, such as can include an end effector 110 such as can include at least one or both of a forceps or one or more electrosurgical electrodes. The wrist 108 mechanism can be driven by one or more pulleys, such as via a ball-and-socket joint in the handle 106 or actuation interface or actuator 112 at or near the handle 106. In the example of FIG. 1, the main body of the handle 106 may remain still or may be grasped or held by the physician or other user and manipulated in three dimensional space in a manner similar to that used in a laparoscopic instrument. The handle 106 can be configured to adds three degrees of freedom of motion of the end effector 110, such as by translating the motion of the surgeon or other user (which can be applied to the actuator 112 via the handle 106) into the small wrist 108 at the distal portion 102 of the telemanipulator device 100.

In FIG. 1, the proximal portion 106 can include a grounding element or front housing 14, from which an extension element or tube 16 or other elongate longitudinal member can extend toward or to form the distal portion 102 of the telemanipulator device 100. The distal portion 102 can be sized, shaped, or otherwise configured to permit it to be inserted into a trocar, such as an 8 millimeter trocar providing a longitudinal lumen with an inner diameter of 8 millimeters, in an example. The housing 14 at the proximal portion 104 can help allow the physician or other user to grossly position the end effector 110 (e.g., such as can include a forceps or other grasper 19, 21 to be positioned or located close to the target location within the patient at which grasping of tissue or another target is desired. The proximal portion 104 can be moved toward and away from the trocar or can move in and out of the trocar and can also pitch up and down and or move left and right pivoting in the trocar. The housing 14 at the proximal portion 104 can include an actuator 112, such as can include a socket 114. The socket 114 can be molded or otherwise formed or configured to be arranged with respect to the handle 106 such as to help provide an additional three degrees of freedom, such as by manipulating the handle 106 with respect to the housing 14 and one or more portions of the actuator 112.

The actuator 112 can include a ball 2, such as which can be sized and shaped and located to mate with the socket 114 of the actuator 112 in the housing 14. This ball 2 and socket 114 joint can help provide an additional three degrees of freedom. The surgeon or other user grasp the housing 14, the handle 106, or both, and can move his or her hands up and down. This will cause the end effector 110 or other distal end of the distal portion 102 to pivotably pitch up and down, such as following the hand motion of the surgeon or other user. By rotating and moving the handle 106 left and right, the surgeon can cause the distal end effector 110 to pivotably follow in the yaw direction. Further, the surgeon or other user can rotate their (human user) wrist about the longitudinal axis defined by the elongate longitudinal tube or member 106. This will induce a roll motion about the longitudinal axis, which, in turn, will be translated via the ball 2 and socket 114 and via the mechanical or electromechanical wrist 108 mechanism to a roll motion of the end effector 110 at the distal end of the distal portion 20 of the telemanipulator device 100.

FIG. 2 shows a close-up view of portions of a mechanical wrist mechanism or manipulation interface 108, showing a pitch axis that intersects the yaw axis at a common intersection point 4 that also intersects a central longitudinal roll axis defined to be coaxial to the elongate member 116. In the example of FIG. 2, the wrist 108 can include a pitch gimbal 202, pivotable about a pitch axis, and a yaw gimbal 204, pivotable about a yaw axis, such as via corresponding respective pullies that can be connected to the ball 2 of the actuator 112 via respective cables. The pitch gimbal 202 and the yaw gimbal 204 can pivotably cooperate with each other such as to allow mutually constrained movement of tube, rod, or other elongate hitch or stem 206 extending from and attached to a core element or ball 208 centered at the common intersection point 4 of the pitch, yaw, and roll axes. The end effector 110 can extend from and be attached to the hitch 206. As shown in FIG. 2, the pitch gimbal 202 and the yaw gimbal 204 can be driven by two wire cables, which can be elastic wires or more rigid wires. The motion of these gimbals 202, 204 can move the ball 208 via the mutually constrained cylindrical tubular or other hitch 206 that moves with these gimbals 202, 204, and which also is capable of rolling about the roll axis extending longitudinally through the device 100 back toward the proximal portion 104 and the actuator 112.

Thus, in FIG. 2 the wrist 108 is governed by three intersecting axes, pitch, roll and yaw which all intersect and coincide at the point 4. All of these mechanisms at or extending from the distal portion 102 of the telemanipulator device 100 can be sized, shaped, and otherwise configured to fit within a cylinder or similar lumen with an inner diameter between 8 mm and 8.5 mm (or even smaller, e.g., 5 millimeters) which will allow insertion into and down a trocar. In FIG. 2, the wrist 108 at the distal portion 102 of the device 100 can include a ball-and-socket joint, such as with the ball 208 constrained by a socket that can be provided in part by the gimbals 202, 208. The ball 208 can be rigidly attached to the tubular hitch 206, which, in turn, can be attached to a base of the end effector 110 such as by being attached to a grasper bottom jaw 19 portion of the end effector 110. The ball 208 with the tubular hitch 206 can be positioned into any orientation using the two gimbals 202, 204. The yaw gimbal 204 pivotably rotates around the yaw axis. The pitch gimbal 202 pivotably rotates around the pitch axis. The yaw and pitch gimbals 202, 204 can be driven by corresponding respective ties or cables 22, such as can be engaged with pulleys on the respective gimbals 202, 204. Both gimbals 202, 204 can include bails such as that can define respective slots through which the tubular hitch 206 can extend and within which the tubular hitch 206 can slide. For example, as the pitch gimbal 202 rotates about the pitch axis, the slot of the pitch gimbal 202 drives the hitch 206, which, in turn, moves the distal portion 102 and the bottom jaw 19 of the end effector 110. The ball 208 can be rotated around the central longitudinal axis that coincides with the main longitudinal shaft of the device 100, such as extending between its proximal portion 104 and its distal portion 102. This central longitudinal axis is the roll axis, which also intersects point 4. Roll may be induced to the grasping jaws of an end effector 110, such as by attaching a flexible torque tube or member extending proximally from the ball 208, such as shown more visibly as torque member 804 in FIG. 8. Rotating the flexible torque tube or member will roll the ball 208, which, in turn, will roll the distal portion and bottom grasper 19, of the end effector 110.

FIG. 3 shows a side view of portions of the device 100, with partial cutaway. In FIG. 3, the distal pitch gimbal 202 and the distal yaw gimbal 204 can respectively include pulleys, such as can be coaxially coincident with their respective pitch and yaw axes. These pulleys can engage with the cables 22, which can extend longitudinally back from the distal portion 102 of the device and the proximal portion 104 of the device 100 to the ball 2 associated with the actuator 112 located with and coupling between the handle 106 and the housing 14 at the proximal portion 104 of the device 100. FIG. 3 shows two cross sectional views C (shown in more detail in FIG. 4) and E (shown in more detail in FIG. 5).

FIG. 4 shows a view taken along the section C-C of FIG. 3. FIG. 4 shows the actuator 112, including the socket 114 and the ball 2. Pulleys 30, 31 can be molded into the ball 2, which can be connected to the handle 106 such as via a connecting member 402. Respective cables 22 can wrap snugly around respective ones of the pulleys 30, 31, which can be molded into a ball feature of the ball 2, which mates with the socket 114 of the actuator 112 at the handle 106 or the housing 14.

FIG. 5 shows a view taken along the section E-E of FIG. 3, in a broken manner so as to illustrate both a proximal portion 104 and a distal portion 102 of the device 100. FIG. 5 shows an example of the pulley system connecting the yaw gimbal pulley 502 of the yaw gimbal 204 via a cable 22 to the corresponding pulley 504 in the ball 2 located at the actuator 112 associated with the handle 106 and the housing 14. The ball and socket surfaces are shown as 32 and 33 in FIG. 5.

FIG. 6 shows an exploded view of an example of the distal wrist 108 mechanism. The ball 208 can be captured in a socket element 36 in a longitudinal member connector 18. Connector 18 can connect the spherical socket 36 to a longitudinal main shaft of the device 100. The two gimbals 202, 204 can respectively include pulleys on at least one end of the gimbal 202, 204. It may be desirable to have pulleys on both ends of one or both of the gimbals 202, 204, such as to help improve traction with the corresponding respective drive cable 22.

FIG. 7 shows another example, such as can include or use one or more sensors or motors to move the pitch gimbal 202 and the yaw gimbal 204. A gyroscopic PC board 708 including a gyroscopic sensor and electronic controller circuitry can be included in the handle 106. For example, a gyroscopic sensor such as Adafruit BNO055 can be used. These sensors are relatively inexpensive. The sensor can be monitored by an inexpensive Arduino micro-controller, such as which can sense the pitch, yaw, and roll of the gyroscopic sensor. By mounting the sensor board 708 in the handle 106, the surgeon can grip the handle 106 and the Arduino micro-controller can sense the orientation of the surgeon's wrist. The Arduino microcontroller can be configured to issue one or more pulse width modulated (PWM) signals, such to respective servomotors 709 and 710, such as which can drive the pitch gimbal 202 and yaw gimbal 204, respectively.

In FIG. 7, the servomotors 709, 710 can respectively include an inexpensive DS3218 20 Kg servomotor. The servomotors 709, 710 respectively drive pulleys 704 and 705 for the yaw gimbal 204 and pitch gimbal 202, respectively. A thin cable 706 can connects respective servo pulleys to the pulleys on the yaw and pitch gimbals, concentric to respective yaw and pitch axes, 700 and 701. These pulleys are included in the respective gimbals, which will drive the ball 208 and tube hitch 206. The electromechanical wrist mechanisms in FIG. 7 were made at 5× scale. The components at 1× scale will fit within an 8 mm trocar. The prototype shown in FIG. 7 provides proof of concept of the coincident yaw, pitch, and roll axes wrist mechanism 108.

FIG. 8 shows an example of portions of a representative mechanical embodiment of the device 100. In this example, the main longitudinal shaft tube and the pitch and yaw gimbals are 2× size (16 mm tube diameter versus 8 mm size planned). FIG. 8 shows an example that can include a bipolar electrosurgical forceps dissector with 2× scale features at the distal end (except the forceps jaws themselves are shown at 1×). The ball 2 can includes two internal, molded pulleys (not visible in this view), such as which, via respective cables, can drive the respective pitch and yaw pulleys that can be respectively located on the pitch and yaw gimbals at a more distal portion of the device 100. The cables connecting the pulleys are omitted from the view of FIG. 8 for viewing clarity, but are understood to be included from the other FIGS. The black hypotube 802, such as can extend through the ball 2, is free to rotate in such manner as to permit the handle 806 to drive the ball 2 up/down (pitch) or left/right (Yaw). The flexible torque tube 804 can be attached to the handle 106 so when the surgeon turns their wrist, the jaws of the end effector 110 will turn (roll). Also, a fourth cable can extend between the jaw pulley and the lever pulley triggered by squeezing together the “scissor-handle” portions of the handle 806. This fourth cable can run through the flexible torque tube 804 that drives roll. The end effector forceps jaws can include active bipolar coagulating jaws, such as can be actuated by a coagulation activation button. To supply power to the electrosurgical electrodes, a power cord may extend from the bottom of the handle. The example shown in FIG. 8 may be a fully disposable version. Another example can include a partially reusable handle with disposable jaw and main shaft component.

### Additional Notes

The following, non-limiting examples, detail certain aspects of the present subject matter to solve the challenges and provide the benefits discussed herein, among others.

Example 1 is a handheld medical intervention device, comprising: a central portion for establishing a reference position of the handheld treatment device; a handle arranged proximal to the central portion and having at least two degrees of rotational freedom relative to the central portion; a distal portion arranged distal to and extending from the central portion; and a manipulation interface operably coupling the distal portion to the central portion, the distal portion being operable by the handle via the manipulation interface to provide at least two degrees of rotational freedom of the distal portion relative to the central portion, wherein: the at least two degrees of rotational freedom of the distal portion include two degrees of rotational freedom that: correspond with two of the at least two degrees of rotational freedom of the handle; and are about a first set of at least two orthogonal axes that intersect at a first point.

In example 2, the subject matter of example 1 optionally includes wherein the handle comprises three degrees of rotational freedom relative to the central portion.

In example 3, the subject matter of example 2 optionally includes wherein the three degrees of rotation freedom of the handle are about a second set of at least three orthogonal axes that intersect at a second point.

In example 4, the subject matter of example 2 optionally includes wherein the first set of at least two orthogonal axes comprises three orthogonal axes that intersect at the first point and the distal portion comprises three degrees of rotational freedom about the first set of three orthogonal axes.

In example 5, the subject matter of any one or more of examples 1-4 optionally includes wherein motion of the distal portion about one of the two axes of the first set of at least two orthogonal axes is independent of motion about the other of the two axes.

In example 6, the subject matter of any one or more of examples 1-5 optionally includes wherein the manipulation interface comprises a core element arranged between a pair of gimbals.

In example 7, the subject matter of example 6 optionally includes wherein the core element is centered at the first point.

In example 8, the subject matter of example 7 optionally includes wherein the core element comprises a spherical bushing.

In example 9, the subject matter of example 7 optionally includes wherein the core element comprises a hoop.

In example 10, the subject matter of any one or more of examples 1-9 optionally includes a torque element extending from the handle to the manipulation interface and having a longitudinal axis, wherein a first angular displacement of the torque element about the longitudinal axis is the same as a second angular displacement of the distal portion about a respective longitudinal axis thereof and regardless of the orientation of the distal portion about other axes.

Example 11 is a handheld medical intervention device, comprising: a central portion for establishing a reference position of the handheld medical device; a handle arranged proximally to the central portion; a distal portion arranged distal to and extending from the central portion; and a manipulation interface coupling the distal portion to the central portion and operable by the handle to control the orientation of the distal portion, wherein a ratio of a first range of motion of the distal portion about a first axis to a second range of motion of the handle about a second axis corresponding to the first axis is less than 1.

In example 12, the subject matter of example 11 optionally includes wherein the handle is operably coupled to the manipulation interface with a pair of spaced apart tensile elements extending along the central portion on opposing sides of a longitudinal axis and the distance between the spaced apart tensile elements is less at the handle than at the manipulation interface.

In example 13, the subject matter of example 12 optionally includes wherein the pair of spaced apart tensile elements comprise adjacent segments of a same cord with first and second ends, the first and second ends being secured to an actuation interface and the cord wrapping around a pulley on the manipulation interface.

In example 14, the subject matter of example 12 optionally includes wherein a first end of each tensile element is secured to an actuation interface and a second opposite end is secured to the manipulation interface.

In example 15, the subject matter of example 14 optionally includes a cam surface on each of the actuation interface and the manipulation interface, the cam surface being defined by a radius equal to half of the distance between the pair of tensile elements at the respective actuation interface and manipulation interface.

In example 16, the subject matter of any one or more of examples 11-15 optionally include wherein the handle is equipped with a positional sensor configured to measure a degree of rotation about the second axis.

In example 17, the subject matter of example 16 optionally includes wherein the manipulation interface is operable by the handle via a servo, the servo adapted to control rotation of the distal portion about the first axis based on a fraction of the degree of rotation of the handle about the second axis.

Example 18 is a handheld medical intervention device, comprising: a central portion for establishing a reference position of the handheld medical device; a handle arranged proximal to the central portion; a distal portion arranged distal to and extending from the central portion; and a manipulation interface arranged on a distal end of the central portion, coupling the distal portion to the central portion, and operable by the handle to control the orientation of the distal portion, wherein the handle is operably coupled to the manipulation interface by a mechanical coupling, the mechanical coupling configured to absorb hand tremor motion of the handle.

In example 19, the subject matter of example 18 optionally includes wherein the mechanical coupling comprises elastic materials for absorbing hand tremor motion of the handle.

In example 20, the subject matter of any one or more of examples 18-19 optionally include wherein the mechanical coupling is controlled by motors adapted to attenuate hand tremor motion of the handle.

Example 21 is a handheld medical intervention device, comprising: a central portion for establishing a reference position of the handheld medical device; a handle arranged proximal to the central portion and having at least two degrees of rotational freedom relative to the central portion and about respective axes of a first set of orthogonal axes that intersect at a first point; a distal portion arranged distal to and extending from the central portion; and a manipulation interface coupling the distal portion to the central portion and operable by the handle to provide at least two degrees of rotational freedom of the distal portion relative to the central portion about respective axes of a second set of orthogonal axes that intersect at a second point.

In example 22, the subject matter of example 21 optionally includes

wherein the second set of orthogonal axes correspond with the first set of orthogonal axes.

In example 23, the subject matter of any one or more of examples 21 and 22 optionally includes wherein the first set of orthogonal axes and the second set of orthogonal axes each comprise three orthogonal axes that intersect at the respective first and second points.

In example 24, the subject matter of any one or more of examples 21-23 optionally includes wherein motion of the distal portion corresponds to motion of the handle.

In example 25, the subject matter of example 24 optionally includes wherein motion of the distal portion corresponds to motion of the handle by a factor less than 1.

In example 26, the subject matter of any one or more of examples 1-25 optionally includes a power source for powering an electrode.

In example 27, the subject matter of example 26 optionally includes wherein the power source is a battery.

In example 28, the subject matter of example 27 optionally includes wherein the battery is arranged on or in the handle.

In example 29, the subject matter of example 27 optionally includes wherein the electrode is arranged on the distal portion and is in selective electrical communication with the battery.

In example 30, the subject matter of example 26 with respect to example 17 optionally includes wherein the servo or motor is in electrical communication with the battery.

In example 31, the subject matter of any one or more of examples 1-30 optionally includes wherein the device is a non-dominant hand instrument.

In example 32, the subject matter of any one or more of examples 1-31 optionally includes wherein the distal portion comprises a pair of jaws.

In example 33, the subject matter of example 32 optionally includes wherein at least one jaw of the pair of jaws includes at least one degree of rotational freedom relative to the distal portion.

In example 34, the subject matter of example 33 optionally includes wherein each jaw of the pair of jaws has a degree of rotational freedom relative to the distal portion.

Example 35 is a handheld endoscopic or laparoscopic device for partial insertion into an orifice of or incision in a patient for treating a target within the patient, the device comprising: a distal portion, sized and shaped for insertion into the patient; a proximal portion, connected to the distal portion, the proximal portion including an external handle for a physician or other user to hold and support the proximal portion during insertion of the distal portion into the patient; a mechanical or electromechanical wrist, located at the distal portion, the wrist providing yaw, roll, and pitch motions about corresponding yaw, roll, and pitch axes, wherein each of the yaw, roll, and pitch axes are arranged to intersect one another; an actuator, located at the handle, the actuator coupled to the wrist for manipulating the wrist for providing the yaw, roll, and pitch motions; an end effector, at the distal portion of the device and distal to the wrist, the end effector providing at least yaw, roll, and pitch motions via the wrist in response to manipulation of the actuator by the user.

In example 36, the subject matter of example 35 optionally includes wherein the wrist includes a first ball, at the distal portion of the device, coupling the actuator to the end effector.

In example 37, the subject matter of any one or more of examples 35-36 optionally includes a second ball, at the proximal portion of the device, coupling the handle to the wrist.

In example 38, the subject matter of example 37 optionally includes wherein the second ball is included in a ball-and-socket at the proximal portion of the device, and wherein the second ball is coupled to the first ball to provide movement of the first ball in response to movement of the second ball.

In example 39, the subject matter of example 38 optionally includes wherein the second ball is coupled to the first ball to provide reduced movement of the first ball in response to and relative to the movement of the second ball.

In example 40, the subject matter of any one or more of examples 37-39 optionally includes wherein the second ball is larger than the first ball.

In example 41, the subject matter of any one or more of examples 35-40 optionally includes at least one of a filter or damping mechanism between the actuator and at least one of the wrist or the end effector to help at least one of smooth or attenuate tremor or noise in application of a force to the actuator by the user.

In example 42, the subject matter of any one or more of examples 35-41 optionally includes wherein the wrist includes: a first gimbal, coupled to the actuator, the first gimbal arranged to pivot about the yaw axis; a second gimbal, coupled to the actuator, the second gimbal arranged to pivot about the pitch axis; and wherein the pitch axis intersects the yaw axis.

In example 43, the subject matter of example 42 optionally includes a rod member, constrained by and movable with the pivoting of each of the first gimbal and the second gimbal, wherein the rod member couples at least a portion of the end effector to the wrist.

In example 44, the subject matter of example 43 optionally includes wherein the rod member couples the at least a portion of the end effector to a first ball included in the wrist.

In example 45, the subject matter of example 43 optionally includes wherein the rod member couples a grasper bottom jaw of the end effector to the wrist.

In example 46, the subject matter of any one or more of examples 35-44 optionally includes wherein the end effector includes a forceps.

In example 47, the subject matter of any one or more of examples 35-46 optionally includes wherein the end effector includes a bipolar electrosurgical electrode.

In example 48, the subject matter of any one or more of examples The device of any one or more of examples 42-47 optionally includes a first cable, coupling a first pulley of the first gimbal to a second ball included in the actuator; and a second cable, coupling a second pulley of the second gimbal to the second ball included in the actuator.

In example 49, the subject matter of any one or more of examples 35-48 optionally includes a torque transmission member coupling the actuator to the wrist for manipulating the wrist for providing the roll motion.

In example 50, the subject matter of example 49 optionally includes wherein the torque transmission member is coupled to a first ball portion of the wrist and defines a coaxial roll axis that intersects with the pitch and yaw axes.

In example 51, the subject matter of any one or more of examples 35-50 optionally includes wherein the actuator comprises: a servomotor, coupled to the wrist; a gyroscopic of other sensor, to receive user input, and to provide a sensor output; and controller, coupled to the sensor and the servomotor, the controller configured to operate the servomotor to control at least one movement of the wrist.

In example 52, the subject matter of example 51 optionally includes wherein the at least the handle and the actuator is user-attachable and user-detachable from at least the distal portion of the device including the wrist and the end effector.

In example 53, the subject matter of any one or more of examples 51-52 optionally includes at least one of an electronic filter or damping mechanism between the actuator and at least one of the wrist or the end effector to help at least one of smooth or attenuate tremor or noise in application of a force to the actuator by the user.

Example 54 is a handheld endoscopic or laparoscopic device for partial insertion into an orifice of or incision in a patient for treating a target within the patient, the device comprising: a distal portion, sized and shaped for insertion into the patient; a proximal portion, connected to the distal portion, the proximal portion including an external handle for a physician or other user to hold and support the proximal portion during insertion of the distal portion into the patient; a ball, located at the distal portion, the ball rotatable to provide yaw, roll, and pitch motions about corresponding yaw, roll, and pitch axes; an actuator, located at the handle, the actuator coupled to the ball for manipulating the ball for providing the yaw, roll, and pitch motions; and an end effector, at the distal portion of the device and distal to the ball, the end effector providing at least yaw, roll, and pitch motions via the wrist in response to manipulation of the actuator by the user.

Example 55 is a handheld endoscopic or laparoscopic device for partial insertion into an orifice of or incision in a patient for treating a target within the patient, the device comprising: a distal portion, sized and shaped for insertion into the patient; a proximal portion, connected to the distal portion, the proximal portion including an external handle for a physician or other user to hold and support the proximal portion during insertion of the distal portion into the patient; a first ball, located at the distal portion, the ball rotatable to provide motion to a more distal end effector; and a second ball, located more proximal than the first ball, the second ball coupled to the first ball to provide reduced rotational movement of the first ball with respect to greater rotational movement of the second ball; and an end effector, movable in response to reduced rotational movement of the first ball in response to the greater rotational movement of the second ball.

Example 56 is a method of using a handheld endoscopic or laparoscopic device for partial insertion into an orifice of or incision in a patient for treating a target within the patient, the method comprising: inserting a distal portion of the device into the patient; grasping a handle to support a proximal portion of the device during insertion of the distal portion into the patient; and actuating, at the handle, an end effector at the distal portion of the device, via a mechanical or electromechanical wrist, located at the distal portion, the wrist providing yaw, roll, and pitch motions about corresponding yaw, roll, and pitch axes, wherein each of the yaw, roll, and pitch axes are arranged to intersect one another.

Example 57 is a method of using a handheld endoscopic or laparoscopic device for partial insertion into an orifice of or incision in a patient for treating a target within the patient, the method comprising: inserting a distal portion of the device into the patient; grasping a handle to support a proximal portion of the device during insertion of the distal portion into the patient; and actuating, at the handle, an end effector at the distal portion of the device, via a ball located at the distal portion, the ball providing yaw, roll, and pitch motions about corresponding yaw, roll, and pitch axes, wherein each of the yaw, roll, and pitch axes are arranged to intersect one another.

In example 58, the subject matter of example 57 optionally includes wherein the actuating comprises: rotating a second ball, located more proximal than the first ball, the second ball coupled to the first ball to provide reduced rotational movement of the first ball with respect to greater rotational movement of the second ball.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A handheld medical intervention device, comprising:
a central portion for establishing a reference position of the handheld treatment device and having a first longitudinal axis;
a handle arranged proximal to the central portion and three degrees of rotational freedom relative to the central portion;
a distal portion arranged distal to and extending from the central portion and having a second longitudinal axis; and
a manipulation interface operably coupling the distal portion to the central portion, the distal portion being operable by the handle via the manipulation interface to pivot about a first point arranged on the first and the second longitudinal axis and with three degrees of rotational freedom about a first set of three orthogonal axes, wherein the second longitudinal axis passes through the first point during pivoting about each of the axes of the first set of three orthogonal axes and all combinations thereof.

2. (canceled)

3. The device of claim 1, wherein the three degrees of rotation freedom of the handle are about a second set of at least three orthogonal axes that intersect at a second point.

4. (canceled)

5. The device of claim 1, wherein motion of the distal portion about one of the three axes of the first set of three orthogonal axes is independent of motion about the other of the three axes.

6. The device of claim 1, wherein the manipulation interface comprises a core element arranged between a pair of gimbals.

7. The device of claim 6, wherein the core element is centered at the second point.

8. The device of claim 7, wherein the core element comprises a spherical bushing.

9. The device of claim 7, wherein the core element comprises a hoop.

10. The device of claim 1, further comprising a torque element extending from the handle to the manipulation interface and having a longitudinal axis, wherein a first angular displacement of the torque element about the first longitudinal axis is the same as a second angular displacement of the distal portion about the second longitudinal axis and regardless of the orientation of the distal portion about other axes.

11. The device of claim 1, wherein a ratio of a first range of motion of the distal portion about a first axis to a second range of motion of the handle about a second axis corresponding to the first axis is less than 1.

12. The device of claim 11, wherein the handle is operably coupled to the manipulation interface with a pair of spaced apart tensile elements extending along the central portion on opposing sides of a longitudinal axis and the distance between the spaced apart tensile elements is less at the handle than at the manipulation interface.

13. The device of claim 12, wherein the pair of spaced apart tensile elements comprise adjacent segments of a same cord with first and second ends, the first and second ends being secured to an actuation interface and the cord wrapping around a pulley on the manipulation interface.

14. The device of claim 12, wherein a first end of each tensile element is secured to an actuation interface and a second opposite end is secured to the manipulation interface.

15. The device of claim 14, further comprising a cam surface on each of the actuation interface and the manipulation interface, the cam surface being defined by a radius equal to half of the distance between the pair of tensile elements at the respective actuation interface and manipulation interface.

16. The device of claim 11, wherein the handle is equipped with a positional sensor configured to measure a degree of rotation about the second axis.

17. The device of claim 16, wherein the manipulation interface is operable by the handle via a servo, the servo adapted to control rotation of the distal portion about the first axis based on a fraction of the degree of rotation of the handle about the second axis.

18. A handheld medical intervention device, comprising:
a central portion for establishing a reference position of the handheld medical device;
a handle arranged proximal to the central portion;
a distal portion arranged distal to and extending from the central portion; and
a manipulation interface arranged on a distal end of the central portion, coupling the distal portion to the central portion, and operable by the handle to control the orientation of the distal portion, wherein the handle is operably coupled to the manipulation interface by a mechanical coupling, the mechanical coupling configured to absorb hand tremor motion of the handle,
wherein the mechanical coupling comprises at least one of:
elastic materials for absorbing hand tremor motion of the handle; or
motors adapted to attenuate hand tremor motion of the handle.

19-20. (canceled)

21. A handheld medical intervention device, comprising:
a central portion for establishing a reference position of the handheld medical device and having a first longitudinal axis;
a handle arranged proximal to the central portion and having at least two degrees of rotational freedom relative to the central portion and about respective axes of a first set of three orthogonal axes that intersect at a first point;
a distal portion arranged distal to and extending from the central portion and having a second longitudinal axis; and
a manipulation interface coupling the distal portion to the central portion and operable by the handle to pivot the distal portion through three degrees of rotational freedom relative to the central portion and about respective axes of a second set of three orthogonal axes that intersect at a second point,
wherein the second longitudinal axis passes through the second point during pivoting about each of the three orthogonal axes of the second set and all combinations thereof.

22. The device of claim 21, wherein the second set of orthogonal axes correspond with the first set of orthogonal axes.

23. (canceled)

24. The device of claim 21, wherein motion of the distal portion corresponds to motion of the handle.

25. The device of claim 24, wherein motion of the distal portion corresponds to motion of the handle by a factor less than 1.

26. The device of claim 1, further comprising a power source for powering an electrode.

27. The device of claim 26, wherein the power source is a battery.

28. The device of claim 27, wherein the battery is arranged on or in the handle.

29. The device of claim 27, wherein the electrode is arranged on the distal portion and is in selective electrical communication with the battery.

30. The device of claim 26, further comprising a servo or motor in electrical communication with the battery.

31. The device of claim 1, wherein the device is a non-dominant hand instrument.

32. The device of claim 31, wherein the distal portion comprises a pair of jaws.

33. The device of claim 32, wherein at least one jaw of the pair of jaws includes at least one degree of rotational freedom relative to the distal portion.

34. The device of claim 33, wherein each jaw of the pair of jaws has a degree of rotational freedom relative to the distal portion.
